(19) United States
(12) Patent Application Publication
(10) Pub. No.: US 2014/0379302 A1
(43) Pub. Date: Dec. 25, 2014

(54) APPARATUS AND METHOD FOR CONTROLLING WATER QUALITY SENSOR FAULTS

(71) Applicant: Electronics and Telecommunications Research Institute, Daejeon-si (KR)

(72) Inventor: Hyeon PARK, Daejeon-si (KR)

(73) Assignee: Electronics and Telecommunications Research Institute, Daejeon-si (KR)

(21) Appl. No.: 14/247,716

(22) Filed: Apr. 8, 2014

(30) Foreign Application Priority Data
Jun. 20, 2013 (KR) 10-2013-0071221

(51) Int. Cl. G01N 33/18; G06F 17/30

(52) U.S. Cl. CPC G01N 33/18; G06F 17/30386
USPC 702/183

(57) ABSTRACT

In a method for controlling water quality sensor faults by receiving sensing data reported from one or more water quality sensors in an apparatus for controlling water quality sensor faults, the method includes: detecting an outlier value in sensing data reported from the one or more water quality sensors; and in response to detection of an outlier value in the sensing data, determining whether a water quality sensor corresponding to the detected outlier value is faulty.

### CROSS-REFERENCE TO RELATED APPLICATION(S)

This application claims priority to and the benefit of Korean Patent Application No. 10-2013-0071221, filed on Jun. 20, 2013, in the Korean Intellectual Property Office, the entire disclosure of which is incorporated herein by reference for all purposes.

### BACKGROUND

1. Field

The following description relates to a technology for controlling water quality sensors, and more particularly, to an apparatus and method for controlling water quality sensor faults by using collaborative information between nodes for managing water quality evaluations based on a ubiquitous sensor network (USN) installed in small to medium-sized streams.

2. Description of the Related Art

Recently, water quality has been evaluated in real time via USN-based sensors directly installed in streams, mainly in small to medium-sized streams. However, management of water quality environment through the USN-based sensors merely relates to collecting and storing water quality data by establishing sensors, a network, and a database. Further, a network of USN-based water quality management system is controlled in a passive manner that only notifies a user of management information regarding overall network equipment.

In such a water quality management system, a network to be managed is not specified, such that when a fault occurs in network constituent elements, the fault may not be diagnosed accurately, and follow-up measures may not be taken automatically, thereby making real time management difficult.

Further, management of network constituent elements tends to check only whether there is a fault in equipment, such that when a fault occurs, the fault may not be diagnosed accurately or promptly, and real-time measures may not be taken properly in response to the fault. As an object of the USN-based water quality management system is to measure and estimate water quality accurately, if faults in network constituent elements (e.g. failures in water quality sensors) cannot be accurately diagnosed in real time, accuracy in measuring and estimating water quality may also be reduced.

In addition, as such fault diagnosis is conducted only through network management, if errors in water quality data cannot be detected due to impurities attached to a water quality sensor, or if appropriate measures cannot be taken in real time due to insufficient information exchange between sensor nodes equipped with sensors, reliability in collected data may be reduced.

### SUMMARY

There is provided an apparatus and method for evaluating water quality in the USN-based water quality management system installed in small to medium-sized streams, in which a fault in a water quality sensor (turbidity sensor or dissolved oxygen sensor) is estimated in conjunction with a water quality evaluator, which obtains a sensing data outlier value automatically without involvement of a user, and the estimated fault in a water quality sensor is accurately diagnosed and controlled by using collaborative information between sensor nodes in a network, and results thereon are provided to a user. As a result, real-time measures may be taken in response to a fault in network elements, and reliable information about water quality may be provided.

According to an exemplary embodiment, there is provided a method for controlling water quality sensor faults by receiving sensing data reported from one or more water quality sensors in an apparatus for controlling water quality sensor faults, the method includes: detecting an outlier value in sensing data reported from the one or more sensors; and in response to detection of an outlier value in the sensing data, determining whether a water quality sensor corresponding to the detected outlier value is faulty.

According to another exemplary embodiment, there is provided an apparatus for controlling water quality sensor faults, the apparatus includes: a water quality evaluator configured to detect an outlier value in sensing data reported from one or more water quality sensors; and a sensor fault determiner, which in response to detection of an outlier value in the sensing data, is configured to determine whether a water quality sensor corresponding to the detected outlier value is faulty.

### BRIEF DESCRIPTION OF THE DRAWINGS

FIG. 1 is a block diagram illustrating an example of a system for managing water quality according to an exemplary embodiment.

FIG. 2 is a detailed block diagram illustrating an example of an apparatus for controlling water quality sensor faults according to an exemplary embodiment.

FIG. 3 is a flowchart illustrating an example method of controlling water quality sensor faults according to an exemplary embodiment.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

Hereinafter, an apparatus and method for controlling water quality sensor faults will be described in detail with reference to the accompanying drawings. The following description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. Also, descriptions of well-known functions and constructions may be omitted for increased clarity and conciseness.

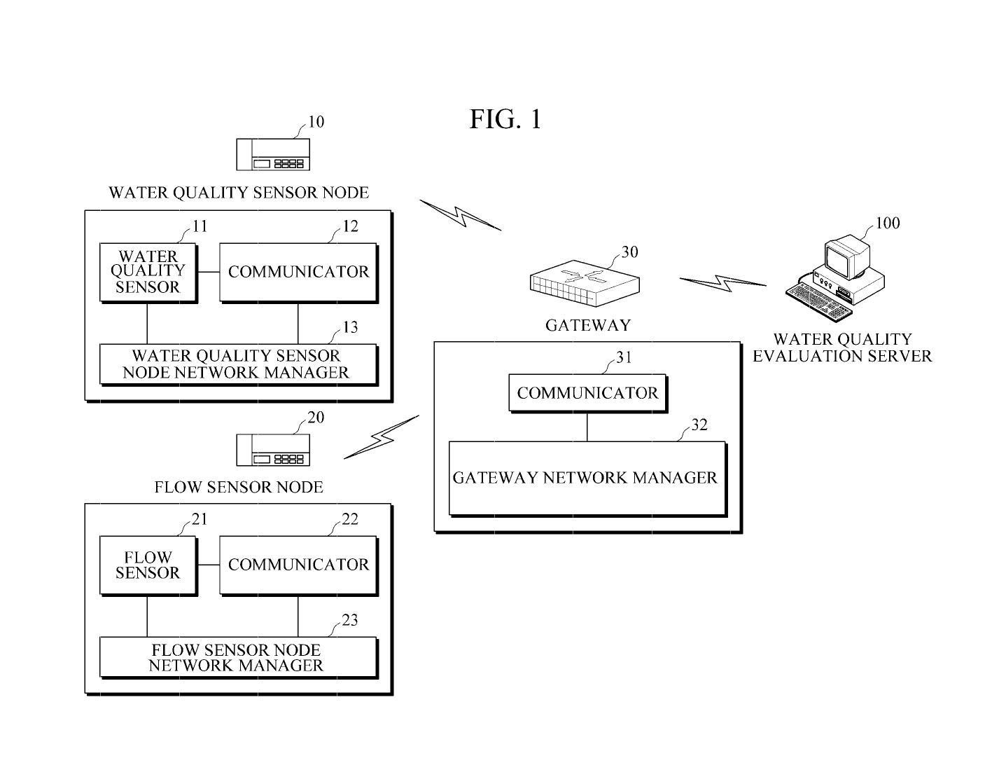

FIG. 1 is a block diagram illustrating an example of a system for managing water quality according to an exemplary embodiment.

Referring to FIG. 1, the system for managing water quality includes a water quality sensor node 10, a flow sensor node 20, a gateway 30, and an apparatus 140 for controlling water quality sensor faults (hereinafter, referred to as “management server”).

The water quality sensor node 10 measures water quality of a certain location to provide the measured water quality for the management server 100 through the gateway 130, and more specifically, the water quality sensor node 10 includes a water quality sensor 11, a communicator 12, and a water quality sensor node network manager 13.

Although not illustrated in FIG. 1, the water quality sensor 11 includes one or more various individual sensors including a turbidity sensor, a dissolved oxygen sensor, and the like, which are necessary for water quality tests, and further includes a water quality sensor main body, a water quality sensor interface board, a water quality sensor battery, and a cleaning device controller.

The communicator 12 is a USN communication module, which receives sensor node controlling information from the management server 100 through the gateway 30, or transmits water quality sensing data measured by the water quality sensor 11 to the management server 100 through the gateway 30.

The water quality sensor node network manager 13 controls the water quality sensor 11 based on sensor node controlling information received from the management server 100 through the communicator 12. According to an exemplary embodiment, the water quality sensor node network manager 13 operates a cleaning device or controls the sensing data to be transmitted.

The flow sensor node 20 measures water flow of a specific location to provide the measured flow to the management server 100 through the gateway 130. More specifically, the flow sensor node 20 includes a flow sensor 21, a communicator 22, and a flow sensor node network manager 23.

Although not illustrated in FIG. 1, the flow sensor 21 includes a sensor necessary for measuring water flow, a flow sensor main body, a flow sensor interface board, and a flow sensor battery. The communicator 22 is a USN communication module, which receives sensor node controlling information from the management server 100, or transmits flow sensing data measured by the flow sensor 21 to the management server 100 through the gateway 30. The flow sensor node network manager 23 controls the flow sensor 21 based on the sensor node controlling information received from the management server 100.

The gateway 30 includes a self-controlled gateway, an RF control, and a USN communicator 31 for transmitting sensing data received from each sensor node, and a gateway network manager 32 that controls management data in conjunction with the USN communicator 31.

The management server 100 analyzes the received sensing data to analyze, estimate, and identify water quality distribution for real-time management. According to an exemplary embodiment, the management server 100 checks network faults of the water quality sensor node 10 so that management is conducted in real time.

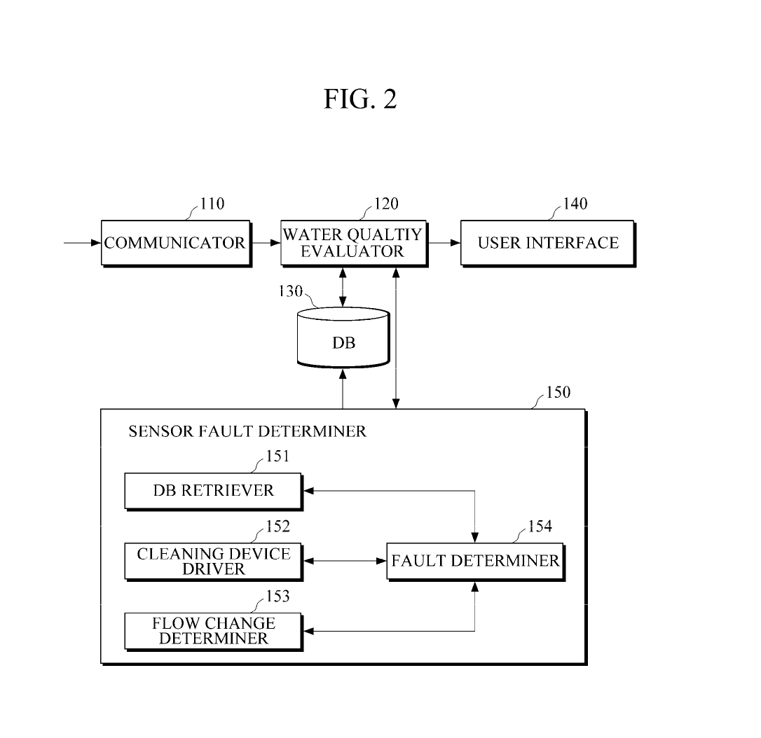

FIG. 2 is a detailed block diagram illustrating an example of an apparatus for controlling water quality sensor faults according to an exemplary embodiment.

Referring to FIG. 2, a management server 100 specifically includes a communicator 110, a water quality evaluator 120, a database 130, a user interface 140, and a sensor fault identifier 150.

The communicator 110 is a USN communication module, which receives sensing data from the water quality sensor node 10 and the flow sensor node 20 through the gateway 30, and transmits controlling information of the water quality sensor node 10 and the flow sensor node 20.

The water quality evaluator 120 receives sensing data transmitted from the water quality sensor node 20 to output the received sensing data to a user through the user interface 140, or stores the received sensing data in the database (DB) 130. Further, according to an exemplary embodiment, in response to detection of an outlier value in turbidity sensing data, the water quality evaluator 120 reports a sensor fault to the sensor fault identifier 150 to make a request to check whether a sensor is faulty.

The database 130 stores sensing data transmitted from the water quality sensor node 10 or the flow sensor node 20, and fault information, including faults of each sensor.

The user interface 140 is a display for outputting water quality evaluation data and information on sensor node faults to a user, and may further include an input for receiving setting information on water quality evaluation system from a user.

The sensor fault identifier 150 checks whether a sensor is faulty in response to a report on turbidity sensor fault from the water quality evaluator 120. To this end, the sensor fault identifier 150 includes a DB retriever 151, a cleaning device driver 152, a flow change determiner 153, and a fault determiner 154.

The DB retriever 151 retrieves the DB 130 to determine whether a sensor fault has already been reported in response to a turbidity sensor fault report from the water quality evaluator 120. That is, the DB retriever 151 retrieves information as to whether there is a water quality interface fault, a water quality main body fault, and/or a water quality sensor turbidity probe fault.

The cleaning device driver 152 operates a cleaning device in the water quality sensor node 10 for a predetermined time, and then outputs a monitoring result, in which the cleaning device is operated for a predetermined number of times for a predetermined period of time, and monitoring results thereof may be output.

The flow change determiner 153 controls the flow sensor node 10 to determine a sudden change in flow. Flow is estimated based on a sensing value of a flow sensor and a sensing value of a depth-of-water sensor obtained from the flow sensor node network manager 23 through the flow sensor 21. That is, the flow change determiner 153 determines whether an outlier value occurs in a previous flow (based on a transmission period of flow information), or in a current flow by using measurement results of outlier values in water flow, in which a flow with a deviation of 10% or more from a previous flow is determined as an outlier.

The fault determiner 154 determines faults based on information output from the DB retriever 151, the cleaning device driver 152, and the flow change determiner 153, reports the information to the water quality evaluator 120.

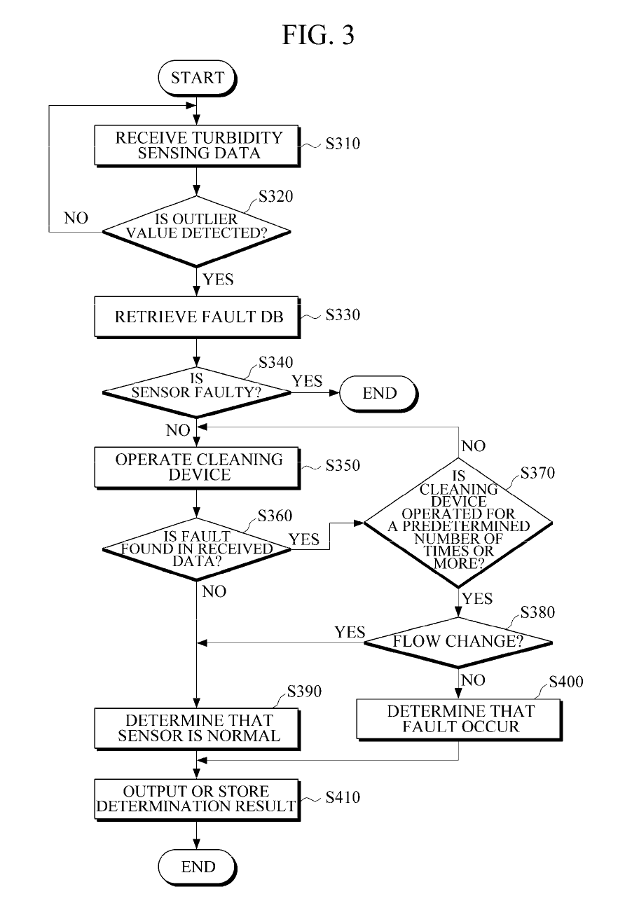

FIG. 3 is a flowchart illustrating an example method of controlling water quality sensor faults according to an exemplary embodiment.

Referring to FIG. 3, the water quality evaluator 110 receives turbidity sensing data in S310 to monitor in S320 whether there is an outlier. Upon monitoring in S320, in response to detection of an outlier value in the turbidity sensing data, the water quality evaluator 120 reports a turbidity sensor fault to the sensor fault identifier 150.

Thereafter, the sensor fault identifier 150 retrieves the DB 130 in S330 to determine in S340 whether a sensor, which is found to have an outlier value in turbidity sensing data, is already reported to be faulty. In other words, by retrieving the DB, a water quality sensor interface fault, a water quality main body fault, and a water quality turbidity probe fault may be detected. Detection of any one fault indicates that a turbidity sensor is already faulty.

Accordingly, in response to a determination in operation S340 that a sensor is already reported to be faulty, the sensor fault identifier 150 ends processing.

However, in response to a determination in S340 that a sensor is not faulty, i.e. the sensor is checked for a water quality main body fault and a water quality sensor turbidity probe fault, and if none of the faults are saved as fault information, the sensor fault identifier 150 operates a cleaning device in S350. That is, even though equipment may not be faulty per se, the sensor fault identifier 150 considers this an actual fault since a turbidity sensor cannot obtain data properly due to impurities attached to the sensor, and operates the cleaning device once to diagnose and verify the faulty situation, and waits for the water quality evaluator 120 to determine an outlier value.

Subsequently, the sensor fault identifier 150 determines whether there is a fault in received data in S360, based on a determination made by the water quality evaluator 120.

In response to a determination in S360 that there is a fault in received data, a cleaning device is operated again for a predetermined number of times. For example, in response to no sensor related fault found in the water quality evaluator before the cleaning device is operated for the third time, it is considered that the sensor has transmitted only normal data and then is restored to a normal state, and the sensor fault identifier 150 reports the normal state of the sensor to a user.

In other words, the sensor fault identifier 150 determines whether a cleaning device has been operated for a predetermined number of times or more in S370. In response to a determination in S370 that the cleaning device has not been operated for a predetermined number of times or more, the sensor fault identifier 150 proceeds to S350. By contrast, in response to a determination in S370 that the cleaning device has operated for a predetermined number of times or more, and for example, an identical sensor fault is consistently reported until the cleaning device is operated for the third time, the sensor fault identifier 150 determines whether there is a change in water flow in S380. That is, in order to determine a sudden flow change, the sensor fault identifier 150 checks whether an outlier value occurs in a previous flow (based on a transmission period of flow information) or in a current flow by using measurement results of outlier values in water flow, and determines a flow with a deviation of 10% or more from a previous flow as an outlier value.

In response to a determination in S380 that there is a sudden change in flow, the sensor fault identifier 150 determines that a fault is not caused by a turbidity probe, but by turbidity sensing data due to flow change in S390. By contrast, in response to a determination in S380 that there is no sudden change in flow, the sensor fault identifier 150 determines there is a sensor fault in S390.

Further, the sensor fault identifier 150 outputs a fault determination result to the water quality evaluator 120, or stores the results in the DB 130.

Although not illustrated in FIG. 3, the water quality evaluator 120 reports a turbidity probe fault to a user through the user interface 140.

In response to a sensor found to be normal after operating a cleaning device, information regarding operation history of the cleaning device is stored. An example of the history information is shown in Table 1 below.

TABLE 1

| Fault | | Cleaning device |
|---|---|---|
| occurrence time | Sensor name | operation count |
| 10:05:02 | Turbidity sensor | 2 |
| 11:10:02 | Turbidity sensor | 2 |
| 12:15:02 | Turbidity sensor | 3 |
| 15:20:02 | Turbidity sensor | 2 |
| 17:20:02 | Turbidity sensor | 2 |

Table 1 shows that, with respect to a fault occurrence in the first row, a sensor returned to a normal state after operating a cleaning device twice, and with respect to a fault occurrence in the third row, a sensor returned to a normal state after operating a cleaning device three times. In such history information, an average ((2+2+3+2+2)/5=2) of an operating count of the cleaning device in response to the latest five fault occurrences is determined as a current operating count of the cleaning device. As a result, when a new fault occurs in an identical sensor, a cleaning device is operated twice at a regular interval, thereby reducing a waiting time for determining an outlier value in the water quality evaluator, and enabling real time processing.

As described above, the apparatus and method for evaluating water quality in the USN-based water quality management system installed in small to medium-sized streams has effects in that a fault in a water quality sensor (turbidity sensor or dissolved oxygen sensor) is estimated in conjunction with a water quality evaluator, which obtains a sensing data outlier value automatically without involvement of a user. An estimated fault in a water quality sensor is accurately diagnosed by using collaborative information between sensor nodes in a network, and is controlled properly by operating network constituent elements, such that a waiting time for determining an outlier value in the water quality evaluator may be reduced, providing real-time processing of faults, and highly reliable water quality data.

A number of examples have been described above. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims. Further, the above-described examples are for illustrative explanation of the present invention, and thus, the present invention is not limited thereto.

What is claimed is:

1. A method for controlling water quality sensor faults by receiving sensing data reported from one or more water quality sensors in an apparatus for controlling water quality sensor faults, the method comprising:
detecting an outlier value in sensing data reported from the one or more water quality sensors; and
in response to detection of an outlier value in the sensing data, determining whether a water quality sensor corresponding to the detected outlier value is faulty.

2. The method of claim 1, wherein the determining whether the water quality sensor corresponding to the detected outlier value is faulty comprises retrieving a database to determine whether the water quality sensor corresponding to the detected outlier value is already reported to be faulty.

3. The method of claim 2, wherein the determining whether the water quality sensor corresponding to the detected outlier value is faulty comprises, in response to retrieving in the database at least one fault among a water quality sensor interface fault, a water quality sensor main body fault, and a water quality sensor turbidity probe fault of the water quality sensor, determining that the water quality sensor is already faulty.

4. The method of claim 2, wherein the determining whether the water quality sensor corresponding to the detected outlier value is faulty comprises:
in response to the water quality sensor not yet being reported to be faulty, operating a cleaning device of the water quality sensor, and monitoring sensing data after a lapse of predetermined time; and
in response to monitoring no fault occurrence, determining that the water quality sensor has returned to a normal state.

5. The method of claim 4, wherein the determining whether the water quality sensor corresponding to the detected outlier value is faulty further comprises, in response to monitoring a fault occurrence, repeating operation of the cleaning device for a predetermined number of times.

6. The method of claim 5, wherein the determining whether the water quality sensor corresponding to the detected outlier value is faulty comprises managing an average operating period of the cleaning device.

7. The method of claim 6, wherein the determining whether the water quality sensor corresponding to the detected outlier value is faulty comprises, in response to monitoring a fault occurrence, using flow sensing data to determine that the water quality sensor is faulty.

8. The method of claim 7, wherein the determining whether the water quality sensor corresponding to the detected outlier value is faulty comprises, in response to no sudden changes in the flow sensing data, determining that the water quality sensor is faulty.

9. An apparatus for controlling water quality sensor faults, the apparatus comprising:
a water quality evaluator configured to detect an outlier value in sensing data reported from one or more water quality sensors; and
a sensor fault determiner, which in response to detection of an outlier value in the sensing data, is configured to determine whether a water quality sensor corresponding to the detected outlier value is faulty.

10. The apparatus of claim 9, wherein the sensor fault determiner comprises:
a database (DB) retriever configured to retrieve a database to determine whether the water quality sensor corresponding to the detected outlier value, is already reported to be faulty;
a cleaning device driver, which in response to the water quality sensor not yet being reported to be faulty, is configured to operate a cleaning device of the water quality sensor and to monitor sensing data after a lapse of predetermined time; and
a flow change determiner, which in response to monitoring a fault occurrence, is configured to use flow sensing data to determine that the water quality sensor is faulty.

11. The apparatus of claim 10, wherein the DB retriever comprises:
retrieving the database, and in response to retrieving at least one fault among a water quality sensor interface fault, a water quality sensor main body fault, and a water quality sensor turbidity probe fault of the water quality sensor, determining that the water quality sensor is already faulty.

12. The apparatus of claim 10, wherein the cleaning device driver is configured to repeat operation of the cleaning device for a predetermined number of times in response to the monitoring of a fault occurrence.

13. The apparatus of claim 10, wherein the cleaning device driver is configured to manage an average operating period of the cleaning device.

14. The apparatus of claim 10, wherein in response to no sudden changes in flow sensing data, the flow change determiner is configured to determine that the water quality sensor is faulty.
